(19) United States
(12) Patent Application Publication
(10) Pub. No.: US 2005/0096696 A1
(43) Pub. Date: May 5, 2005

(54) Arteriotomy closure device with anti-roll anchor

(76) Inventor: Andrew Thomas Forsberg, Minneapolis, MN (US)

Correspondence Address: L. Grant Foster;HOLLAND & HART LLP, 555 - 17th Street, Suite 3200, P.O. Box 8749, Denver, CO, 80201, US

(21) Appl. No.: 10/700,746

(22) Filed: Nov. 4, 2003

(51) Int. Cl. A61D 1/00; A61B 17/08

(52) U.S. Cl. 606/213

(57) ABSTRACT

The present invention is directed to an anchor that is configured to minimize or overcome lateral rotational moments that could potentially cause the anchor to laterally roll out of its intended position during use. The anchor is shaped to allow for a low profile longitudinal position and expanded transverse position. A device is used to initially insert the anchor in the low profile longitudinal position and then longitudinally rotate the anchor into the expanded transverse position when it is inserted through an incision or opening. The anchor is positioned at an internal portion of the incision or opening such that it seals an internal side of the incision. A sealing plug positioned at an external portion of the incision or opening. The sealing plug and anchor are cinched together with a suture to facilitate rapid healing of the incision or opening.

### FIELD OF THE INVENTION

This invention relates to medical devices, and more particularly to a method and apparatus for preventing an anchor from rolling laterally when used with a device for sealing internal tissue incisions or punctures.

### BACKGROUND OF THE INVENTION

Medical science has advanced tremendously in the last century to include the use of numerous complex internal procedures to treat various human conditions. Many of these procedures require a surgeon to puncture or slice into a portion of the internal human anatomy in order to perform a particular process. For example, many cardiology procedures require accessing the internal portion of a corporeal vessel. After the procedure is completed, the surgeon must repair damage to the internal organ or vessel in order for the patient to properly recover. While it is possible to suture or seal the skin and other organs after the procedure, it is not always possible to suture delicate vessels with the same technique. Therefore, new techniques have been developed to seal punctures and incisions in delicate vessels such as arteries and veins.

Closure devices used to seal subcutaneous punctures in delicate vessels in a quick and efficient manner so as to minimize the recovery time of patients undergoing these types of procedures are described in U.S. Pat. Nos. 5,282,827 and 5,662,681, which are hereby incorporated by reference. These closure devices and others are available under the trade name Angio-Sea®. The closure devices and processes associated therewith are commonly used to seal arteriotomys such as the ones created when the femoral artery is deliberately punctured in order to perform a procedure. The femoral artery is often punctured in order to clear blockages or obstructions in the patient's circulatory system. The above-mentioned patents describe embodiments of a puncture closure device in which an anchor is inserted through the arteriotomy and positioned against an interior wall of the artery. A collagen sponge is positioned at an exterior wall of the artery above the arteriotomy. The anchor and collagen sponge are then sandwiched or compressed together to facilitate rapid hemostasis and sealing of the arteriotomy.

One problem often associated with this type of sealing device is that occasionally the anchor rolls laterally after it is placed inside the arteriotomy. If the anchor rolls out of its proper internal position, the arteriotomy may not properly seal between the anchor and the collagen sponge. The lateral rolling is typically caused by a rotational moment generated on the anchor as the suture passes through the anchor and sandwiches the collagen sponge and the anchor across the arteriotomy. Therefore, there is a need for an anchor for use with a tissue puncture closure device that minimized the possibility of lateral rolling when the anchor is positioned internally.

### SUMMARY OF THE INVENTION

The present invention contemplates an anchor that is configured to minimize lateral rotational moments that tend to cause the anchor to laterally roll out of its intended position during use. The anchor is shaped to allow for a low profile longitudinal position and an expanded transverse position. A device such as an insertion sheath is used to initially insert the anchor in the low profile longitudinal position. The anchor then automatically rotates into an expanded transverse position when it is inserted through an incision or opening. The anchor is positioned at an internal portion of a subcutaneous incision or opening such that it seals an internal side of the incision. A sealing plug is positioned at an external portion of the incision or opening. The sealing plug and anchor are compressed together with a suture to sandwich and seal the incision or opening and thus facilitate rapid healing.

One embodiment of the anchor minimizes lateral rotational moments on the anchor by winding a suture around and through the anchor multiple times. The suture is wound such that the forces induced upon the anchor by the suture are substantially balanced by one another with respect to rotational moments. For example, a downward force on one side of the anchor is balanced by an upward force on the same side of the anchor so as to avoid causing lateral rotation of the anchor.

Another embodiment reduces the likelihood of lateral rotation of the anchor by adding an anti-rolling tab. The anti-rolling tab increases the lateral width of the anchor, which in turn means that a larger rotational force is necessary to rotate the anchor out of its intended position.

Another embodiment for minimizing lateral rotational forces on the anchor includes positioning a suture inlet and a suture outlet as close as possible to a longitudinal axis of rotation of the anchor. Lateral rotational forces generated on the anchor are proportional to the distance between a suture inlet or outlet and the longitudinal axis of rotation. Therefore, by minimizing the distance between the suture inlet and outlet and the lateral axis of rotation, the lateral rotational forces are also minimized.

Another embodiment for minimizing lateral rotational forces on the anchor includes beveling or smoothing edges of suture passages through the anchor. The lateral rotational forces induced on the anchor are generally a result of friction between the suture and the anchor when a retraction force is exerted onto the suture. Therefore, by beveling the locations at which the suture engages and slides through the anchor, friction is minimized and consequently the lateral rotational forces are also minimized.

Another embodiment for minimizing lateral rotational forces on the anchor includes lubricating the suture used to compress the anchor and the sealing plug together. By lubricating the suture, friction and resulting lateral rotation forces can be minimized.

The embodiments described above may also be combined in order to create other anchor embodiments that are also less likely to suffer from lateral rotational misplacement during a closure procedure. The foregoing and other features, utilities, and advantages of the invention will be apparent from the following detailed description of the invention with reference to the accompanying drawings.

### BRIEF DESCRIPTION OF THE DRAWINGS

FIG. 1A is a cut-away side view of tissue puncture closure device according to the prior art, wherein an anchor is in a low profile longitudinal configuration for deployment through an arteriotomy;

FIG. 1B is a cut-away side view of the closure device of FIG. 1A, with the anchor rotated to an expanded transverse configuration and positioned on an internal side of the arteriotomy;

FIG. 1C is a side view of the prior art anchor illustrated in FIG. 1B;

FIG. 1D is a top view of the prior art anchor illustrated in FIG. 1B;

FIG. 1E is an end view of the prior art anchor illustrated in FIG. 1B showing rotational forces induced upon the anchor when a retraction force is exerted on a suture extending through the anchor, causing a collagen sponge and anchor to compress together;

FIG. 1F is a cut-away side view of the prior art anchor and collagen sponge illustrated in FIG. 1B showing the anchor in an improper lateral position resulting from the anchor laterally rolling;

FIG. 2A is perspective view of an anchor in accordance with one embodiment of the present invention wherein a single suture is wound around and through the anchor to balance out the rotational forces induced on the anchor when the anchor is compressed together with a collagen sponge;

FIG. 2B is a perspective view of the anchor illustrated in FIG. 2A showing a free body diagram of the forces on the anchor according to the winding of FIG. 2A;

FIG. 3 is a perspective view of an anchor in accordance with an alternative embodiment of the present invention in which an anti-roll tab is incorporated with the anchor to prevent the anchor from rolling inside an arteriotomy;

FIG. 4 is perspective view of an anchor in accordance with another embodiment of the present invention in which the suture is attached to the anchor in a manner that minimizes rotational forces;

FIG. 5A is a side view of an anchor in accordance with another embodiment of the present invention in which the edges of the suture inlet and suture outlet are beveled to minimize frictional forces and thereby reduce the overall rotational moment induced on the anchor when the anchor is compressed together with the collagen sponge; and

FIG. 5B is a sectional view of the anchor illustrated in FIG. 5A showing the beveling on the suture inlet and outlet.

### DETAILED DESCRIPTION OF THE INVENTION

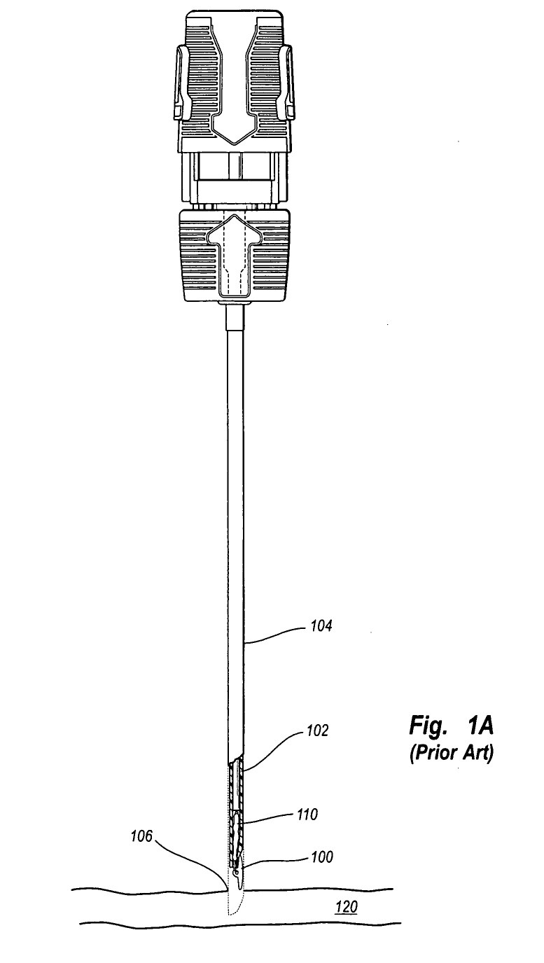

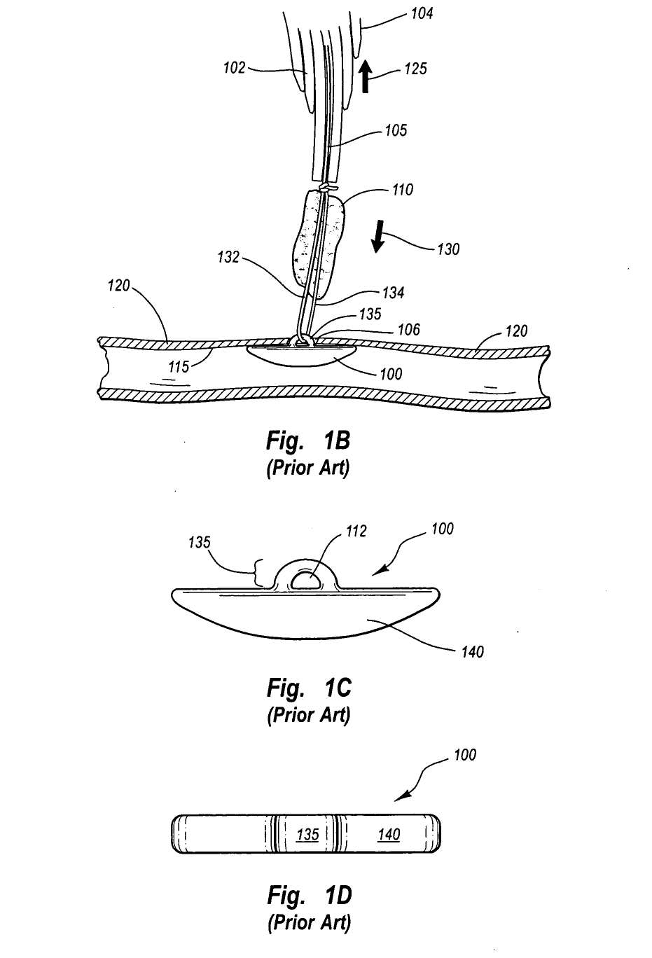

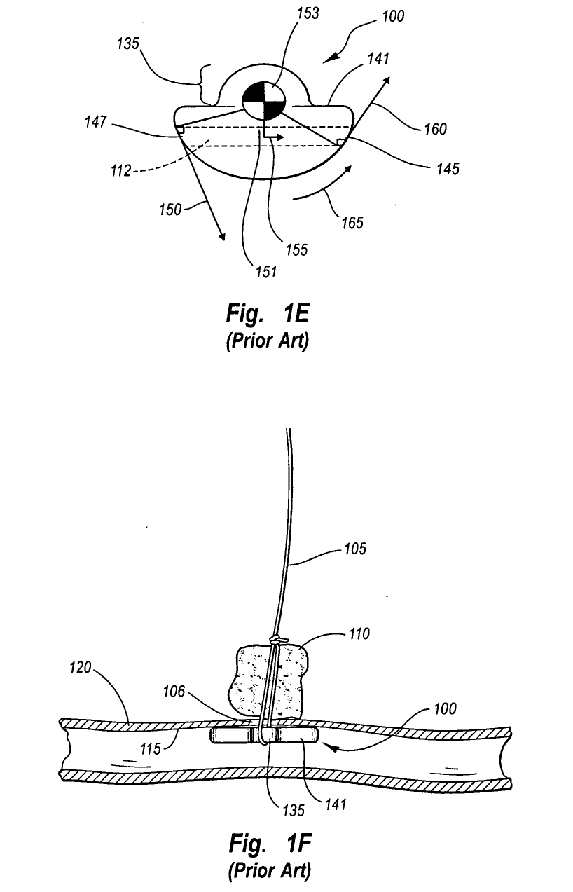

FIGS. 1A-1F illustrate a prior art tissue puncture closure device with a rotateable anchor. The prior art anchor is designated generally at 100. FIG. 1A illustrates the anchor 100 in a low profile longitudinal configuration resting outside of a carrier tube 102. The anchor 100 is substantially aligned with the carrier tube 102 in the low profile configuration shown. The carrier tube 102 and anchor 100 are shown inserted into an insertion sheath 104, which holds the anchor in its low profile configuration aligned with the carrier tube 102 and is partially inserted through a subcutaneous puncture 106 in an artery 120. In order to seal the puncture 106, the anchor 100 passes through the puncture 106 and the insertion sheath 104, and into the artery 120. When the anchor 100 exits the insertion sheath 104, it is no longer held in the low-profile configuration and it automatically rotates into an expanded transverse configuration as shown in FIG. 1B. The automatic rotation of the anchor 100 into the expanded transverse configuration shown in FIG. 1B may also be facilitated by retracting the carrier tube 102 with respect to the insertion sheath 104.

FIG. 1B illustrates the anchor 100 positioned against an internal wall 115 of the artery 120. A sealing plug, which according to the present embodiment is a collagen sponge 110, is positioned external to the artery 120. The anchor 100 and the collagen sponge 110 are coupled together with a suture 105 such that an upward force 125 or retraction of the suture 105 causes the collagen sponge 110 to move distally 130 toward the anchor 100, where the collagen sponge 110 and anchor 100 sandwich and seal the puncture 106. The suture 105 extends down through the collagen sponge 110, in through the anchor 100, and back up to the collagen sponge 110 where it is fixably secured to the collagen sponge 110 or slip-knotted onto itself. A portion of the suture 105 extending into the anchor 100 is designated as a suture anchor inlet portion 132 and a portion of the suture 105 extending out from the anchor 100 is designated as a suture anchor outlet portion 134.

FIGS. 1C and 1D illustrate a side and top view, respectively, of the anchor 100 illustrated in FIG. 1B. The anchor 100 includes a surface nub 135, an internal suture passageway 112, and a body 140. FIG. 1E is an end view of the anchor 100 and illustrates rotational forces induced upon the anchor 100 when an upward force 125 is exerted onto the suture 105 as shown in FIG. 1B. The rotational forces include several forces, consolidated for simplicity in FIG. 1E at an inlet 147, an outlet 149, and an internal surface 151 where the suture 105 (FIG. 1B) slides across the suture passageway 112. A first consolidated force 150 is a frictional force resulting from the suture anchor inlet portion 132 (FIG. 1B) rubbing against the anchor 100 as it passes down through the anchor 100. A second consolidated force 160 is also a frictional force, resulting from the suture anchor outlet portion 134 (FIG. 1B) rubbing against the anchor 100. A third consolidated force 155 is a frictional force as well and results from the friction caused by the suture 105 (FIG. 1B) as it passes through the suture passageway 112 and rubs against the internal surface 151 of the anchor 100. A rotational moment 165 is created by the combined effect of the forces 150, 155, 160 spaced a distance from a longitudinal axis of rotation 153 of the anchor 100. The rotational moment 165 causes the anchor 100 to occasionally roll out of position when compressed towards the collagen sponge 100 by the suture 105.

FIG. 1F illustrates the position of the anchor 100 and the collagen sponge 110 after the anchor rolls out of its proper position. When the anchor 100 rolls out of the proper position, the puncture or arteriotomy in the artery 120 is not properly sealed between the anchor 100 and the collagen sponge 110. A top surface 141 of the anchor 100 is designed to contact internal wall 115 of the artery 120, and the nub 135 is designed to insert partially into the puncture 106 to ensure proper location of the anchor 100. When the anchor 100 rolls laterally, a convex rounded side portion of the anchor 100 is likely to contact the internal wall 115, and the nub 135 becomes useless, causing a less-than-ideal seal of the puncture 106, which may not heal as efficiently as it otherwise could.

Therefore, embodiments of the present invention reduce or eliminate the possibility of lateral rolling of an anchor. Accordingly, reference is made below to drawings that illustrate presently preferred embodiments of the invention, which may be used to replace the anchor 100 in the apparatus described above. It is to be understood, however, that the drawings are diagrammatic and schematic representations of the presently preferred embodiments, and are not limiting of the present invention, nor are they necessarily drawn to scale.

The present invention relates to an anchor that is configured to minimize or overcome lateral rotational moments that tend to cause the anchor to roll laterally out of its intended position during use. The anchor of the present invention is shaped to allow for a low profile longitudinal position and an expanded transverse position. An insertion sheath or other device is used to initially insert the anchor in the low profile longitudinal position, and the anchor automatically rotates longitudinally into the expanded transverse position when it is inserted through an incision or opening or when a suture is retracted. The anchor is positioned at an internal portion of an incision or opening such that it seals the internal side of the incision. A collagen sponge is also positioned at an external portion of the incision or opening. The collagen sponge and anchor are compressed together with a suture to seal the incision and to facilitate rapid healing. If the anchor is allowed to laterally roll when it is in the expanded transverse position, the anchor may not properly seal the internal side of the incision. Also, while embodiments of the present invention are described in the context of a method and apparatus for minimizing or overcoming lateral rotational moments on an anchor used with a puncture closure device, it will be appreciated that the teachings of the present invention are applicable to other applications as well.

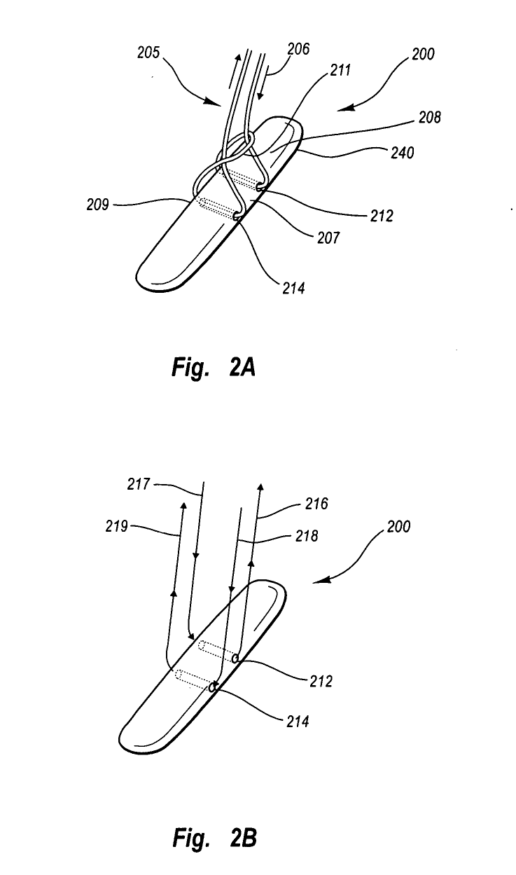

Referring next to FIGS. 2A and 2B, a first embodiment of an anchor 200 and suture 205 according to principles of the present invention is shown. As shown in FIG. 2A, the suture 205 is laterally wound around and through the anchor 200 in a manner that minimize potential rotational moments induced on the anchor 200 during use. More specifically, the suture 205 rigging is arranged to substantially balance some of the forces caused by the suture 205 as it passes through the anchor 200, and therefore reduce or eliminate rotational moments. For example, as shown in FIG. 2A, the suture 205 passes distally from the collagen sponge 110 (FIG. 1A) to the anchor 200 in a direction indicated by arrows 206 adjacent to the suture 205. The suture 205 traverses a first or upper surface 208 and passes through a first suture passageway 212 disposed in the anchor 200. The suture 205 enters the first suture passageway 212 on a first side 207 in a first direction and exits the first suture passageway 212 on a second side 209 opposite of the first side 207. The suture 205 then loops around itself and enters a second suture passageway 214. However, the suture enters the second passageway through the second side 209 in a second direction and exits at the first side 207. The suture then extends proximally back toward the collagen sponge 110 (FIG. 1A).

The suture 205 rigging through both the first and second suture passageways 212, 214 in opposite directions results in a plurality of forces on the anchor 200 shown in a free-body diagram in FIG. 2B. As shown in FIG. 2B, a first set of forces 216, 217 generated as the suture 205 (FIG. 2A) passes through the first suture passageway 212 is offset and substantially balanced by a second set of forces 218, 219 generated as the suture 205 (FIG. 2A) passes through the second suture passageway 214. However, it is likely that the first and second sets of forces 216, 217, 218, 219 will not be perfectly balanced. For example, force 216 and force 219 may be larger than force 217 and force 218 such that the anchor 200 tends to move in the direction of forces 216 and 219. However, the first and second sets of forces 216, 217, 218, 219 are substantially balanced with respect to forces that cause a rotational moment. It will be understood by those of skill in the art having the benefit of this disclosure, however, that other suture 205 rigging configurations similar to or quite different from the configuration shown in FIGS. 2A and 2B may also be used to offset and balance forces on the anchor 200 and therefore minimize rotational moments that tend to cause the anchor 200 to roll laterally when the suture 205 is pulled to sandwich the anchor 200 and the collagen sponge 110 (FIG. 1B) across an arteriotomy.

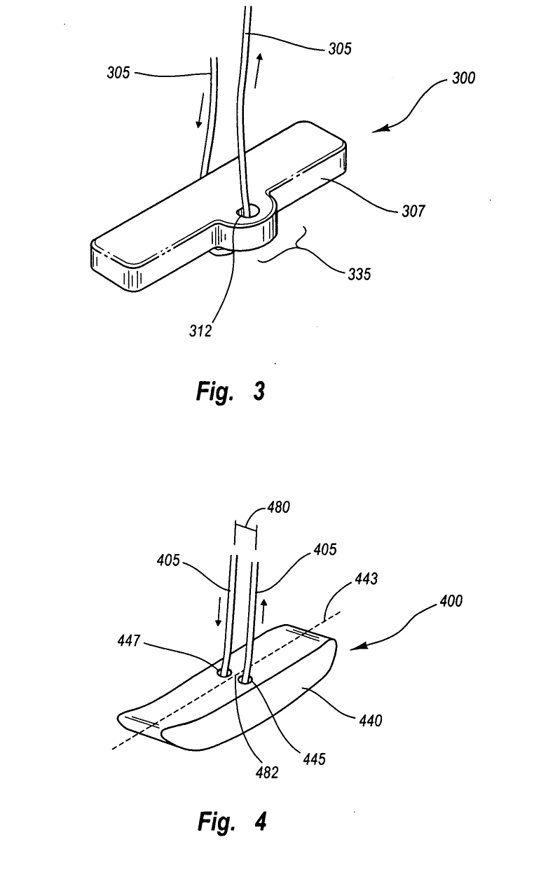

Reference is next made to FIG. 3, which illustrates another embodiment of an anchor 300 for limiting lateral rolling as a result of rotational moments. According to the embodiment of FIG. 3, the anchor 300 includes an anti-rolling tab 335. The anti-rolling tab 335 is a single tab that extends laterally from a first side 307 of the anchor 300. By incorporating the anti-rolling tab 335 with a conventional or modified anchor, the anchor 300 is substantially prevented from laterally rolling. The addition of the anti-rolling tab 335, which is intended to bear against the internal wall 115 (FIG. 1B) of the artery 120 (FIG. 1B), means that a larger lateral rotational moment is required before the anchor 300 will roll. Moreover, according to some embodiments two or more anti-rolling tabs are included with the anchor 300 to provide additional lateral support. The anchor 300 illustrated in FIG. 3 also illustrates a suture 305 extending around the anchor 300 and up through a suture passageway 312. Alternatively, the suture passageway 312 may be placed in a different location without affecting the anti-rolling features of this embodiment. The frictional forces generated between the anchor 300 and the suture 305 are not as critical in this embodiment because of the anti-rolling tab 335. Therefore, the anchor 300 is substantially prevented from laterally rolling following insertion into an artery and compression against an internal wall of the artery.

Reference is next made to FIG. 4, which illustrates yet another embodiment of an anchor 400 in which lateral rotational forces are minimized by spacing a suture inlet 447 and suture outlet 445 apart by a minimized distance 480. The minimized distance 480 may be related to the structural integrity of the composition of the anchor 400. That is, if the anchor 400 is comprised of a stronger material, the minimized distance is shorter than if the anchor 400 is comprised of a weaker material. The minimized distance 480 can be shortened to any length as long as a bridge 482 between the inlet 447 and the outlet 445 maintains its integrity.

According to the embodiment of FIG. 4, the suture inlet 447 and suture outlet 445 are longitudinally aligned and equally spaced from a longitudinal axis of rotation 443. By minimizing the spacing 480 between the suture inlet 447 and suture outlet 445, the distance between the suture inlet and outlets 447, 445 and the longitudinal axis 443 is reduced. The magnitude of the moments induced upon the anchor 400 as the suture 405 slides therethrough is proportional to the spacing of the suture inlet 447 and the suture outlet 445 from the longitudinal axis 443 (see FIG. 1E). Therefore, by reducing the spacing of the suture inlet and outlet 447, 445 from the longitudinal axis 443, rotational moments induced on the anchor 400 when a suture 405 slides therethrough are also reduced.

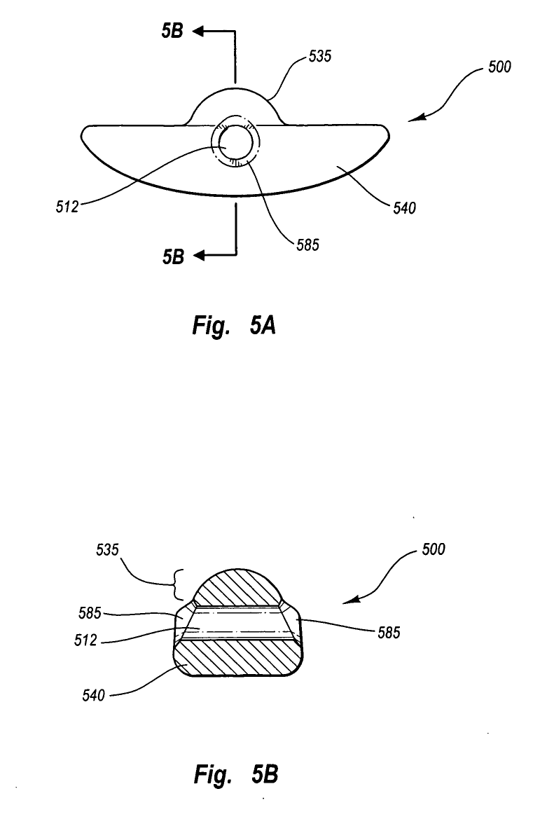

Reference is next made to FIGS. 5A and 5B, which illustrate an anchor 500 in accordance with another embodiment of the present invention. FIG. 5A illustrates a side view and FIG. 5B illustrates a sectional view taken along a section line shown in FIG. 5A. The anchor 500 includes a surface nub 535, a suture passageway 512, and a body 540. In this embodiment, edges 585 of the suture passageway 512 are beveled to minimize frictional forces and thereby reduce the overall rotational moment induced on the anchor 500 when the anchor 500 is compressed together with the collagen sponge 110 (FIG. 1B). The rotational moment induced upon the anchor 500 when the suture 105 (FIG. 1B) slides through the suture passage 512 is proportional to the friction between the suture and the anchor 500. Therefore, beveling or otherwise smoothing the edges 585 at the suture passage 512 entrance and exit reduces the magnitude of frictional rotational forces. Alternatively or in conjunction with the embodiment of FIGS. 5A and 5B, the suture 105 (FIG. 1B) may also be lubricated to minimize the frictional rotational forces between the suture 105 (FIG. 1B) and the anchor 500.

While this invention has been described with reference to certain specific embodiments and examples, it will be recognized by those skilled in the art that many variations are possible without departing from the scope and spirit of this invention. For example, the teachings of one embodiment may be combined with the teachings of another and remain consistent with the scope and spirit of this invention. The invention, as defined by the claims, is intended to cover all changes and modifications of the invention, which do not depart from the spirit of the invention. The words “including” and “having,” as used in the specification, including the claims, shall have the same meaning as the word “comprising.”

1. An internal tissue puncture sealing device, comprising:
a suture;
a rotateable anchor shaped to advance in a low profile longitudinal configuration and to automatically rotate into an expanded transverse configuration when inserted through the internal tissue puncture, wherein the rotateable anchor is configured to minimize lateral rotational moments in the expanded transverse configuration;
a sealing plug connected to the rotateable anchor via the suture such that the suture extends through the sealing plug, through the rotateable anchor, and back to the sealing plug.

2. An internal tissue puncture sealing device according to claim 1 wherein the sealing plug is positioned at an external portion of an arteriotomy.

3. An internal tissue puncture sealing device according to claim 1 wherein the rotateable anchor and sealing plug are configured to compress against one another forming a seal across an internal and external portion of the internal tissue puncture, respectively.

4. An internal tissue puncture sealing device according to claim 1 wherein the suture extends through the rotateable anchor and sealing plug such that when a retracting force is applied to the suture, the rotateable anchor and sealing plug compress together.

5. An internal tissue puncture sealing device according to claim 1 wherein the rotateable anchor comprises a first suture passageway and a second suture passageway, and wherein the suture is looped through the first passageway in a first direction and through the second passageway in a first direction for offsetting rotational moments on the anchor in response to a force applied to the suture.

6. An internal tissue puncture sealing device according to claim 1, further comprising at least one beveled suture passageway disposed in the anchor.

7. An internal tissue puncture sealing device according to claim 1 wherein the suture is lubricated.

8. An internal tissue puncture sealing device according to claim 1 wherein the anchor comprises a suture passageway having an inlet and an outlet, and wherein the inlet and outlet are spaced a minimal distance apart from one another while maintaining structural integrity of the suture passageway.

9. An internal tissue puncture sealing device according to claim 1 wherein the anchor further comprises an anti-rolling tab extending from a side thereof.

10. An arteriotomy sealing device, comprising:
a suture;
an anchor shaped to advance in a low profile longitudinal configuration and to automatically rotate into an expanded transverse configuration when retracted, wherein the suture is looped through the anchor multiple times such that forces generated on the anchor upon retraction of the suture are substantially balanced with respect to rotational moments;
a sealing plug connected to the anchor via the suture such that the suture extends through the sealing plug, through the anchor, and back to the sealing plug.

11. An arteriotomy sealing device according to claim 10 wherein the anchor further comprises at least two suture passageways through which the suture extends, the at least two suture passageways spaced in a complimentary manner from a longitudinal axis of rotation of the anchor such the forces generated at the at least two suture passages are substantially balanced with respect to rotational moments upon retraction of the suture.

12. An arteriotomy sealing device according to claim 11 wherein the at least two suture passageways comprises only two suture passageways, and wherein each suture passageway is laterally spaced from the longitudinal axis of rotation by a substantially equal distance.

13. An arteriotomy sealing device according to claim 12 wherein the suture extends through a first of the two suture passageways in a first direction, loops around itself, and extends through a second of the two suture passageways in a second direction.

14. An arteriotomy sealing device according to claim 11, wherein at least one of the at least two suture passageways is beveled and the suture is lubricated.

15. An arteriotomy sealing device according to claim 12 wherein the anchor further comprises an anti-roll tab extending from a side thereof.

16. A tissue puncture closure device, comprising:
a suture;
a rotateable anchor shaped to advance in a low profile longitudinal configuration and to automatically rotate into an expanded transverse configuration upon insertion through the tissue puncture, wherein the rotateable anchor includes at least one anti-rolling tab positioned to prevent the rotateable anchor from rolling in a lateral manner;
a sealing plug connected to the rotateable anchor via the suture such that the suture extends through the sealing plug, through the rotateable anchor, and back to the sealing plug.

17. A tissue puncture closure device according to claim 16 wherein the at least one anti-rolling tab comprises a single tab that extends laterally from a side of the rotateable anchor such the rotateable anchor is prevented from rolling laterally when positioned against an internal wall of an artery.

18. A tissue puncture closure device according to claim 16 wherein the anchor further comprises at least one suture passageway.

19. A tissue puncture closure device according to claim 18 wherein the at least one suture passageway is beveled.

20. A tissue puncture closure device according to claim 18 wherein the anchor comprises at least two suture passageways spaced in a complimentary manner from a longitudinal axis of rotation of the anchor such that forces generated at the at least two suture passages are substantially balanced with respect to rotational moments upon retraction of the suture.

21. An internal tissue puncture closure device according to claim 18 wherein the at least one suture passageway comprises an inlet and an outlet, and wherein the inlet and outlet are spaced a minimal distance apart from one another while maintaining structural integrity of the suture passageway.

22. A tissue puncture closure device, comprising:
a suture;
a rotateable anchor shaped to advance in a low profile longitudinal configuration and to automatically rotate into an expanded transverse configuration when inserted into the tissue puncture, wherein the rotateable anchor includes a suture inlet and a suture outlet, and wherein the suture inlet and suture outlet are spaced from one another by a minimized distance so as to minimize lateral rotational moments placed upon the rotateable anchor by passing the suture therethrough;
a sealing plug connected to the rotateable anchor via the suture such that the suture extends through the sealing plug, through the rotateable anchor via the suture inlet and suture outlet, and back to the sealing plug.

23. A tissue puncture closure device according to claim 22 wherein the suture is lubricated.

24. A tissue puncture closure device according to claim 22 wherein one or both of the suture inlet and suture outlet are beveled.

25. A tissue puncture closure device according to claim 22, further comprising an anti-roll tab extending from a side of the anchor.

26. An arteriotomy sealing device, comprising:
a suture;
a rotateable anchor shaped to advance in a low profile longitudinal configuration and to automatically rotate into an expanded transverse configuration when inserted through the arteriotomy, wherein the rotateable anchor includes a suture passageway, and wherein the passageway is beveled to minimize frictional forces induced upon the anchor as the suture passes through the suture passageway;
a sealing plug connected to the rotateable anchor via the suture such that the suture extends through the sealing plug, through the rotateable anchor via the suture passageway, and back toward the sealing plug.

27. An arteriotomy sealing device according to claim 26 wherein the rotateable anchor further comprises an anti-roll tab extending from a side thereof.

28. A tissue puncture closure device, comprising:
a rotateable anchor shaped to advance in a low profile longitudinal configuration and to automatically rotate into an expanded transverse configuration when inserted into the tissue puncture;
a lubricated suture passing through the rotateable anchor;
a sealing plug connected to the rotateable anchor via the lubricated suture such that the lubricated suture extends through the sealing plug, through the rotateable anchor, and back to the sealing plug.

29. A method of sealing a subcutaneous tissue puncture, comprising:
inserting an anchor through the subcutaneous tissue puncture;
minimizing a rotational moment about a longitudinal axis of the anchor;
sandwiching the subcutaneous tissue puncture between the anchor and a sealing plug.

30. A method of sealing a subcutaneous tissue puncture according to claim 29 wherein sandwiching further comprises retracting a suture connected between the anchor and the sealing plug.

31. A method of sealing a subcutaneous tissue puncture according to claim 30 wherein minimizing further comprises providing the anchor with at least two suture passageways spaced in a complimentary manner from a longitudinal axis of rotation of the anchor such that forces generated at the at least two suture passages are substantially balanced with respect to rotational moments upon retraction of the suture.

32. A method of sealing a subcutaneous tissue puncture according to claim 30 wherein minimizing further comprises beveling a suture passageway disposed in the anchor.

33. A method of sealing a subcutaneous tissue puncture according to claim 30 wherein minimizing further comprises lubricating the suture.

34. A method of sealing a subcutaneous tissue puncture according to claim 30 wherein minimizing further comprises providing an anti-roll tab on a side of the anchor.

35. A method of sealing a subcutaneous tissue puncture according to claim 30 wherein minimizing further comprises reducing a spacing between an entrance and exit of a suture passageway disposed in the anchor.
